(19) United States
(12) Patent Application Publication
(10) Pub. No.: US 2007/0093851 A1
(43) Pub. Date: Apr. 26, 2007

(54) Surgical seal

(75) Inventors: Peter Moran, Bramley (GB); Stuart Skeleton Moran, Bramhope (GB)

Correspondence Address: APPLIED MEDICAL RESOURCES CORPORATION, 22872 Avenida Empresa, Rancho Santa Margarita, CA, 92688, US

(73) Assignee: KONINKLIJKE PHILIPS ELECTRONICS N.V., Eindhoven (NL)

(21) Appl. No.: 10/560,886

(22) PCT Filed: Jun. 7, 2004
(86) PCT No.: PCT/GB04/02413

§ 371 Date: Dec. 12, 2005

(30) Foreign Application Priority Data
Jun. 13, 2003 (GB) 0313730.4

(51) Int. Cl. A61F 11/00

(52) U.S. Cl. 606/108

(57) ABSTRACT

A seal for laparoscopic port comprising: a base adapted to engage a cannula, the base including an axial aperture for a surgical instrument; a multiplicity of jaws mounted on the base, the jaws being movable radially with respect to the aperture between an open position wherein a shaft of the surgical instrument may pass freely and a closed position wherein the jaws engage said shaft and provide a restraining force restraining radial movement of the shaft; and an actuator rotatable to urge the jaws to move between said open position and said closed position; wherein the actuator includes a click stop arrangement adapted to provide frictional engagement at a position intermediate the open and closed positions to hold the jaws at the intermediate position

This invention relates to a seal for use with a surgical instrument to provide a gas tight seal through which the instrument may pass. The invention relates particularly but not exclusively to a seal for a laparoscopic port.

WO 01/89397 discloses a surgical seal for a laparoscopic port comprising:

a base adapted to engage a cannula, the base including an axial aperture for a surgical instrument; a multiplicity of jaws mounted on the base, the jaws being movable radially with respect to the aperture between an open position wherein the shaft of the instrument may pass freely and a closed position wherein the jaws engage said shaft to provide a restraining force restraining movement of the shaft; and an actuator rotatable to urge the jaws to move between said open position and said closed position. The disclosure of this specification is incorporated into the present specification by reference.

The jaws of the previously disclosed seal may engage or restrain instruments having shafts of different diameters. The jaws are preferably movable along guides which may comprise channels, tracks or runners. In the preferred embodiment the jaws include a runner adapted to be received in a respective guideway in the actuator, arranged so that rotation of the actuator causes radial movement of the jaws. The guideway comprises an arcuate channel which may have the configuration of a parabolic curve.

The seal also includes a diaphragm adapted to contact the shaft of the surgical instrument and further includes a lip adapted to engage a radially outwardly facing portion of each jaw, so that the diaphragm is forced open as the jaws move to the open position. The aperture of the jaws is continuously adjustable between maximum and minimum positions.

Although continuous variability of the opening of the jaws is beneficial for accommodating a range of surgical instruments and particularly for providing a large aperture for insertion or withdrawal, a fixed intermediate diameter to accommodate and securely engage a commonly used instrument, such as a laparoscope is not provided.

According to a first aspect of the present invention a seal for a laparoscopic port comprises:

a base adapted to engage a cannula, the base including an axial aperture for a surgical instrument;

a multiplicity of jaws mounted on the base, the jaws being movable radially with respect to the aperture between an open position wherein a shaft of the surgical instrument may pass freely and a closed position wherein the jaws engage said shaft and provide a restraining force restraining radial movement of the shaft; and

an actuator rotatable to urge the jaws to move between said open position and said closed position;

wherein the actuator includes a click stop arrangement adapted to provide frictional engagement at a position intermediate the open and closed positions to hold the jaws at the intermediate position.

The click stop arrangement may comprise a discontinuity, preferably a protrusion, for example a rib or button or a recess for example a detent on the actuator arranged to engage a complementary discontinuity, preferably a detent or protrusion on the base. A captive ball bearing extended by a spring and a complementary socket may be used. However, simple construction with fewer parts is preferred for ease of assembly and sterilisation. A plurality of click stop positions may be provided.

Alternatively the click stop arrangement may comprise a protrusion, detent or other formation on the jaw adapted to engage a respective complementary formation on the actuator. Preferably there is a peg, pin or other protrusion on each jaw, and the actuator includes a recess or detent dimensioned to receive and engage the protrusion. A plurality of click stop positions may be provided.

Preferably the jaws are biassed towards their closed position.

Preferred embodiments include a resilient diaphragm having a central aperture adapted to contact the shaft of a surgical instrument, the diaphragm including a lip, each jaw engaging the lip so that the aperture of the diaphragm is forced to open as the jaws move from the closed position towards the open position.

In particularly preferred embodiments a restoring force is provided by the resilient diaphragm. Such an arrangement avoids the need for additional spring or resilient means and ensures that the force applied by each jaw to the shaft of an instrument is the same. This facilitates axial location of the instrument in use.

Furthermore, the restoring force facilitates use of a simple click stop arrangement as it is only necessary to retain the actuator in an open position, because the actuator moves automatically towards the closed position where no manual force is applied to it.

In particularly preferred embodiments each jaw includes:

a follower movable along a respective guide on the actuator,

the guide having inner and outer ends corresponding to open and closed positions of the jaw,

the guide further having an intermediate discontinuity adapted to engage the follower preventing closure of the jaw by providing a closure resisting force greater than said restoring force.

A plurality of discontinuities may be used to provide two or more intermediate click stop positions. The guide may comprise a channel or slot and the follower may comprise a pin, peg or other protrusion. The discontinuity may comprise a recess or detent in the inner surface of the guide into which the protrusion is received and held until manual rotation is applied to the actuator. The recess may be shaped to snugly receive the follower.

Seals in accordance with this invention have the advantage that the jaws and diaphragm may be locked in the fully open position to allow for insertion or removal of articles, but the jaws when released automatically close from the fully open position to the intermediate position. The jaws remain in the intermediate position in the absence of external force from the surgeon. The intermediate position can be selected to allow insertion of a standard size shaft without allowing lateral movement of the shaft after insertion. This arrangement avoids the need for manual intervention to set the diameter of the aperture of the jaws during the surgical procedure.

The actuator may be arranged so that the jaws are fully opened or closed by rotation through an angle of 30-90°, preferably 30-45° so that a surgeon may open or close the jaws using his fingers but without need for rotation of the wrist. Any convenient number of jaws may be provided, preferably at least five, most preferably seven.

According to a second aspect of the present invention there is a provided a method of use of a seal for laparoscopic port as previously described.

The seal as previously described allows the diameter of the aperture of the diaphragm to be adjusted and set prior to insertion of an instrument. Although the jaws can be deflected from their minimum closed position by the tip of an instrument as it is inserted, as with prior art devices, it is advantageous that the diameter can be set to a predetermined intermediate and maximum value. This makes insertion of the instrument easier as less force is required. Also, the instrument is held axially during insertion. Alternatively, the jaws can be set to the maximum open position, the instrument inserted and the jaws released so that the jaws engage and drive the shaft of the instrument without need to set the instrument to a particular diameter of shaft.

The invention is further described by means of example but not in any limitative sense with reference to the accompanying drawings of which:

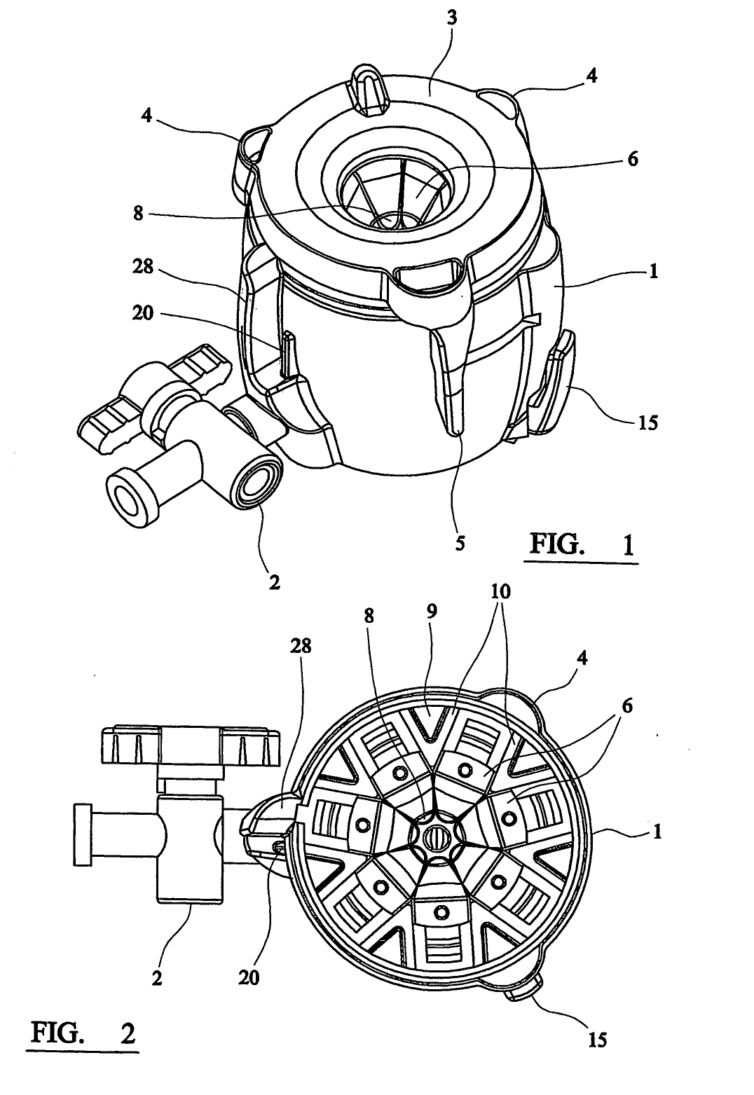

FIG. 1 is a perspective view of a seal in accordance with the invention.

FIG. 2 is a plan view of the seal with the actuator cover removed.

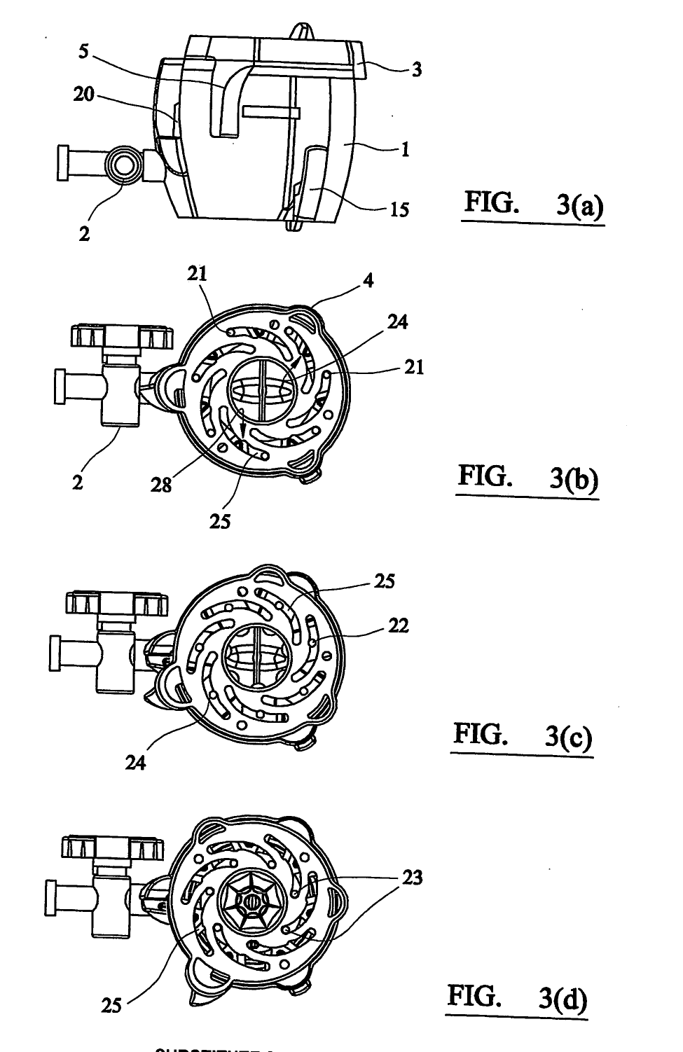

FIG. 3 is an elevation and three sections illustrating operation of the invention.

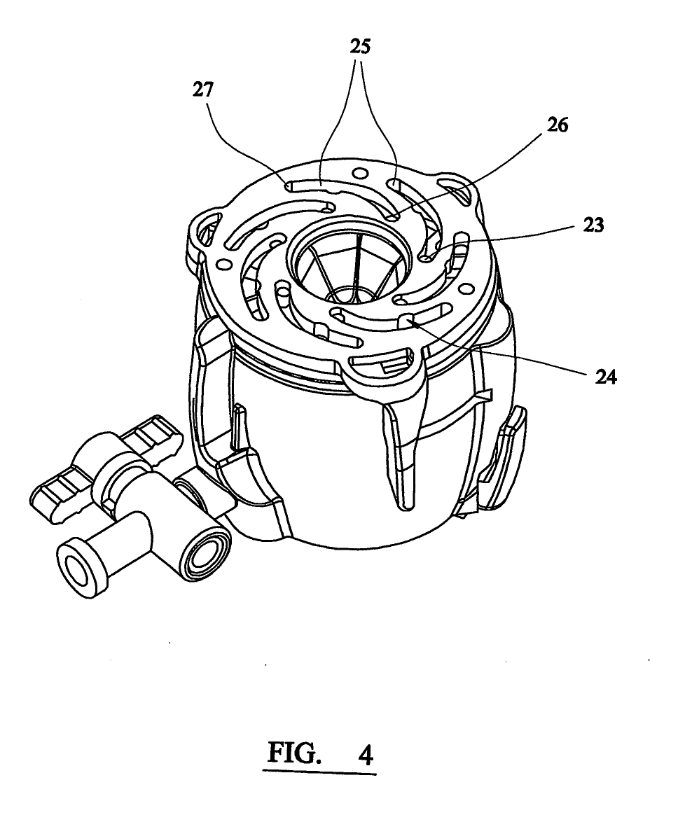

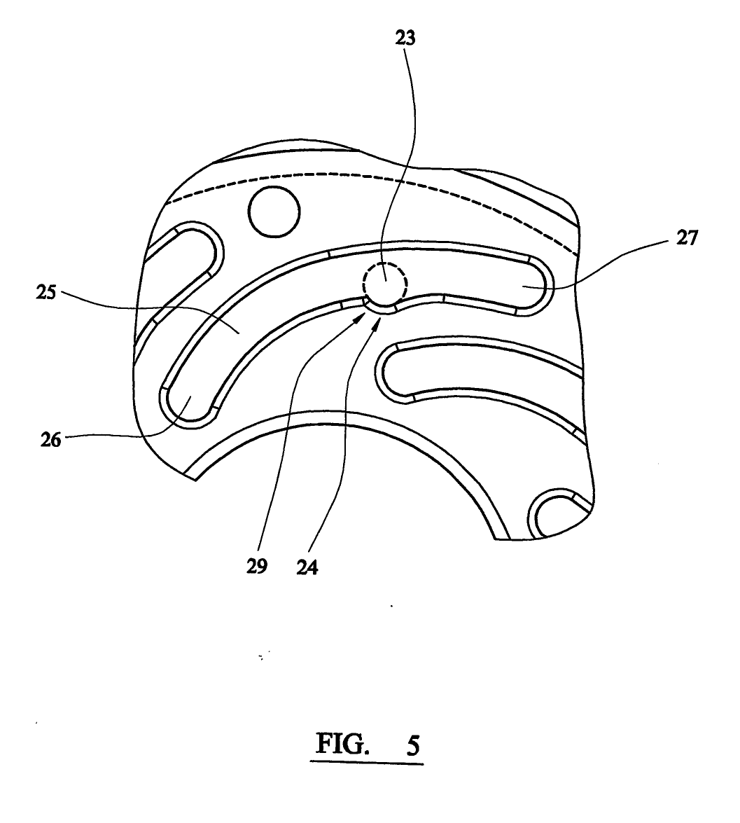

FIG. 4 is an enlarged perspective sectional view.

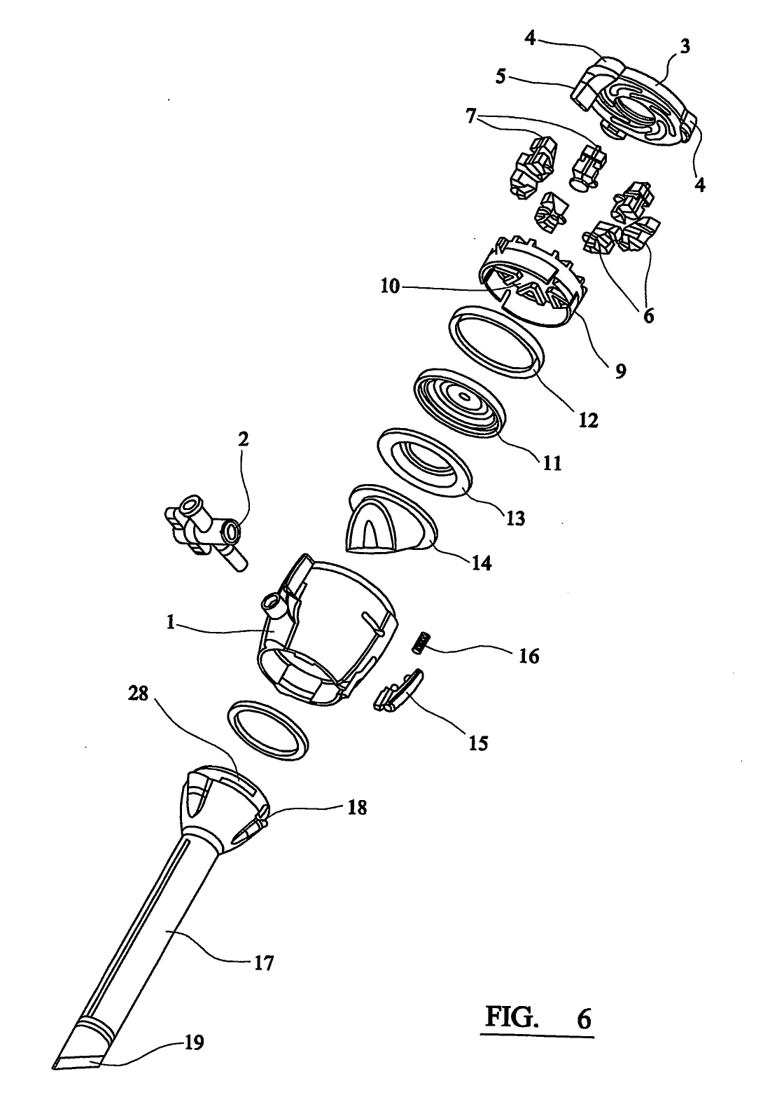

FIG. 5 is a detail of the sectional view and

FIG. 6 is an exploded view of a cannular including a seal in accordance with this invention.

The disclosure of our co-pending WO 01/89397 is referred to. This specification discloses a laparoscopic seal, not having a click stop arrangement. The disclosure of this specification is incorporated into the present specification by reference.

The laparoscopic seal illustrated in the Figures comprises a casing 1 having a rotatable actuator cap 3. Finger grips 4 on the cap facilitate rotation of the latter and a distally extending lever 5 allows the seal to be moved between open and closed positions. Seven radially movable jaws 6 mounted in radial guideways 10 in a carrier plate 9, have proximally extending follower pins or studs 7 which engage sideway channels 25 on the distal surface of the cap 3. The teeth 8 of the jaws 6 cooperate to form an aperture to engage the shaft of the surgical instrument (not shown). When the cap 3 is rotated clockwise the lever 5 engages the protrusion or rib 20, resiliently deforming to pass over the latter to form a click stop end position wherein the jaws are fully opened. A diaphragm 11 is engaged on hook portions on the jaws for example as described with reference to the drawings in WO 01/89397. In this way, the diaphragm is opened as the jaws are opened. The resilient restoring force of the dilated diaphragm urges the jaws into the closed position so that the jaws engage the shaft of an instrument inserted through the seal. The diaphragm 11 also engages the shaft of the instrument forming a gas tight seal. The gasket 11 is received within the distal aperture of carrier plate 9 by a gasket 12. An elastomeric duck billed valve 14 serves to form a conventional fluid type seal when the valve is not in use. The casing 1 may be coupled to a cannula 17 by means of a twist fit arrangement 28. A locking pin 15 driven by spring 16 and an engagement 18 on the cannula form a latch to prevent accidental removal of the valve from the cannula. The cannula has a conventional tip 19 for insertion into the body cavity.

FIG. 3 shows three cross-sectional views through the cap 3 illustrating different positions of the jaws followers or pins as the actuator cap 3 rotates.

In FIG. 3b the actuator cap is rotated fully clockwise so that the jaws are opened and the followers 21 reach the outermost end of the arcuate channels 25. In this position the lever 5 passes over the blocking rib 20 and abuts the abutment 28. The force required to urge the lever 5 anticlockwise over the rib 20 is greater than the restoring force of the resilient diaphragm so that the valve remains open unless the actuator cap is twisted manually.

FIG. 3c shows an intermediate position wherein the followers 22 are received in the recesses 24 on the inner surfaces of the arcuate channels 25. When the actuator cap 3 is released manually from the maximum open position as shown in FIG. 1b, the restoring force of the resilient diaphragm causes the cap to rotate anticlockwise until the followers 22 are received in the recesses 24. The resilient force of the diaphragm engages the followers 22 within the recesses preventing further closure of the valve. In this intermediate position the valve has a standard diameter, for example, 10 mm to receive a laparoscopic camera or other commonly used instrument. Further manual rotation in the anticlockwise direction urges the followers 22 out of the recesses 25 so that the restoring force of the resilient diaphragm can completely close the jaws as shown in FIG. 3d.

The arrangement is shown on a larger scale in FIG. 4.

FIG. 5 shows a single channel 25 having the recess 24 dimensioned to receive the follower pin 23. The recess 24 has a larger radius on the outer surface to allow easy passage of the follower 23, but has a smaller radius on the inner surface 29 to retain the follower in the absence of manual pressure.

The channel 25 has an exponential curvature to facilitate smooth closing of the valve when it is opened slightly and when the resilient diaphragm is only slightly dilated.

1. A seal for laparoscopic port comprising:
a base adapted to engage a cannula, the base including an axial aperture for a surgical instrument;
a multiplicity of jaws mounted on the base, the jaws being movable radially with respect to the aperture between an open position wherein a shaft of the surgical instrument may pass freely and a closed position wherein the jaws engage said shaft and provide a restraining force restraining radial movement of the shaft; and
an actuator rotatable to urge the jaws to move between said open position and said closed position;
wherein the actuator includes a click stop arrangement adapted to provide fictional engagement at a position intermediate the open and closed positions to hold the jaws at the intermediate position.

2. A seal as claimed in claim 1 wherein the click stop arrangement comprises a discontinuity on the actuator arranged to engage a complementary discontinuity on the base.

3. A seal as claimed in claim 2 wherein the discontinuity comprises a
protrusion or recess on the actuator arranged to engage a complementary detent or protrusion on the base.

4. A seal as claimed in claim 1 wherein the click stop arrangement comprises a protrusion or detent on the jaw adapted to engage a complementary formation on the actuator.

5. A seal as claimed in claim 4 wherein the click stop arrangement comprises a pin extending from each jaw, the pin being received in a recess in a guide on the actuator.

6. A seal as claimed in claim 1 wherein each jaw includes a follower movable along a respective guide on the actuator,
the guide having inner and outer ends corresponding to open and closed positions of the jaw,
the guide further having an intermediate discontinuity adapted to engage the follower preventing closure of the jaw by providing a closure resisting force greater than said restoring force.

7. A seal as claimed in claim 5 wherein the guide is an arcuate channel or slot in the actuator.

8. A seal as claimed in claim 7 wherein the channel or slot is parabolic or exponential in shape.

9. A seal as claimed in claim 5 wherein the recess is located on the radial inner surface of the guide.

10. A seal as claimed in claim 1 wherein the jaws are biassed radially inwardly.

11. A seal as claimed in claim 10 wherein the jaws engage a lip of the resilient diaphragm and a biassed radially inwardly when the diaphragm is dilated.

12. A seal as claimed in claim 6 wherein the guide is an arcuate channel or slot in the actuator.
